(19) United States
(12) Patent Application Publication
(10) Pub. No.: US 2013/0109986 A1
(43) Pub. Date: May 2, 2013

(54) ELECTRODE LEAD OF PACEMAKER AND PACEMAKER

(71) Applicants: LIANG LIU, Beijing (CN); CHEN FENG, Beijing (CN); LI QIAN, Beijing (CN); YU-QUAN WANG, Beijing (CN); LI FAN, Beijing (CN); WEN-MEI ZHAO, Beijing (CN)

(72) Inventors: LIANG LIU, Beijing (CN); CHEN FENG, Beijing (CN); LI QIAN, Beijing (CN); YU-QUAN WANG, Beijing (CN); LI FAN, Beijing (CN); WEN-MEI ZHAO, Beijing (CN)

(73) Assignees: HON HAI PRECISION INDUSTRY CO., LTD., Tu-Cheng (TW); Tsinghua University, Beijing (CN)

(21) Appl. No.: 13/527,784

(22) Filed: Jun. 20, 2012

(30) Foreign Application Priority Data
Oct. 28, 2011 (CN) 201110333483.3

(51) Int. Cl. A61N 1/05; A61B 5/04; B82Y 5/00

(52) U.S. Cl. 600/509; 607/119; 977/742; 977/925

(57) ABSTRACT

An electrode lead of a pacemaker includes a lead wire. The lead wire includes at least one sub-lead wire and an electrode head. The sub-lead wire includes a core wire structure, a first insulating layer and a carbon nanotube composite structure. The first insulating layer coats on an outer surface of the core wire structure. The carbon nanotube composite structure is wound around an outer surface of the core wire structure. The electrode head is disposed on an end of the lead wire and electrically connected with the core wire structure of the sub-lead wire. The pacemaker includes a pulse generator and the electrode lead electrically connected to the pulse generator.

This application claims all benefits accruing under 35 U.S.C. §119 from China Patent Application No. 201110333483.3, filed on Oct. 28, 2011, in the China Intellectual Property Office, the contents of which are hereby incorporated by reference. This application is related to common-assigned applications entitled, “METHOD FOR MAKING PACEMAKER ELECTRODE LEAD,” filed**** (Atty. Docket No. US40858); “ELECTRODE LEAD OF PACEMAKER AND PACEMAKER” filed **** (Atty. Docket No. US40859); “PACEMAKERS AND PACEMAKER LEADS” filed **** (Atty. Docket No. US40863); “PACEMAKERS AND PACEMAKER LEADS” filed **** (Atty. Docket No. US40864); “ELECTRODE LEAD OF PACEMAKER AND PACEMAKER USING THE SAME” filed **** (Atty. Docket No. US40865); “ELECTRODE LEAD OF PACEMAKER AND PACEMAKER USING THE SAME” filed **** (Atty. Docket No. US40866); “PACEMAKER ELECTRODE LEAD AND PACEMAKER USING THE SAME” filed **** (Atty. Docket No. US40867); “PACEMAKER ELECTRODE LEAD AND PACEMAKER USING THE SAME” filed **** (Atty. Docket No. US40868).

### BACKGROUND

1. Technical Field

The present disclosure relates to an electrode lead of a pacemaker and a pacemaker using the same.

2. Description of Related Art

A pacemaker is an electronic therapeutic device that can be implanted in living beings such as humans. The pacemaker includes a pulse generator, and an electrode lead. The pulse generator is used to emit a pulsing current, via the electrode lead, to stimulate a diseased organ such as a human heart, to work normally.

The electrode lead usually includes a lead wire made from metal or alloy. However, a mechanical strength and toughness of the lead wire decreases with the diameter. The fierce seizure suffered by a patient or normal activities of the patient may cause damage to the implanted electrode lead. Therefore, a working life of the electrode lead of the pacemaker will decrease, threatening the safety of the patient.

What is needed, therefore, is to provide an electrode lead of a pacemaker which has good mechanical strength and toughness, notwithstanding small physical size, to improve the working life of the electrode lead and the pacemaker.

### BRIEF DESCRIPTION OF THE DRAWING

Many aspects of the present disclosure can be better understood with reference to the following drawings. The components in the drawings are not necessarily to scale, the emphasis instead being placed upon clearly illustrating the principles of the present embodiments.

FIG. 1 is an isometric view of one embodiment of a pacemaker.

FIG. 2 is a schematic view of an embodiment of a sub-lead wire of the pacemaker.

FIG. 3 is a schematic view of an embodiment of a lead wire having a helical structure.

FIG. 4 is a schematic view of an embodiment of a bundle structure composed of a plurality of sub-lead wires compactly arranged in parallel.

FIG. 5 is a schematic view of an embodiment of a twisted wire structure composed of a plurality of sub-lead wires twisted with each other.

FIG. 6 shows a Scanning Electron Microscope (SEM) image of a non-twisted carbon nanotube wire.

FIG. 7 shows an SEM image of a twisted carbon nanotube wire.

FIG. 8 is a schematic view of a carbon nanotube coated with a metal material layer.

FIG. 9 is a transmission electron microscope (TEM) image of the carbon nanotube coated with the metal material layer.

FIG. 10 is a sectional view of an embodiment of an electrode lead.

FIG. 11 is a sectional view of another one embodiment of an electrode lead.

### DETAILED DESCRIPTION

The disclosure is illustrated by way of example and not by way of limitation in the figures of the accompanying drawings in which like references indicate similar elements. It should be noted that references to “another,” “an,” or “one” embodiment in this disclosure are not necessarily to the same embodiment, and such references mean at least one.

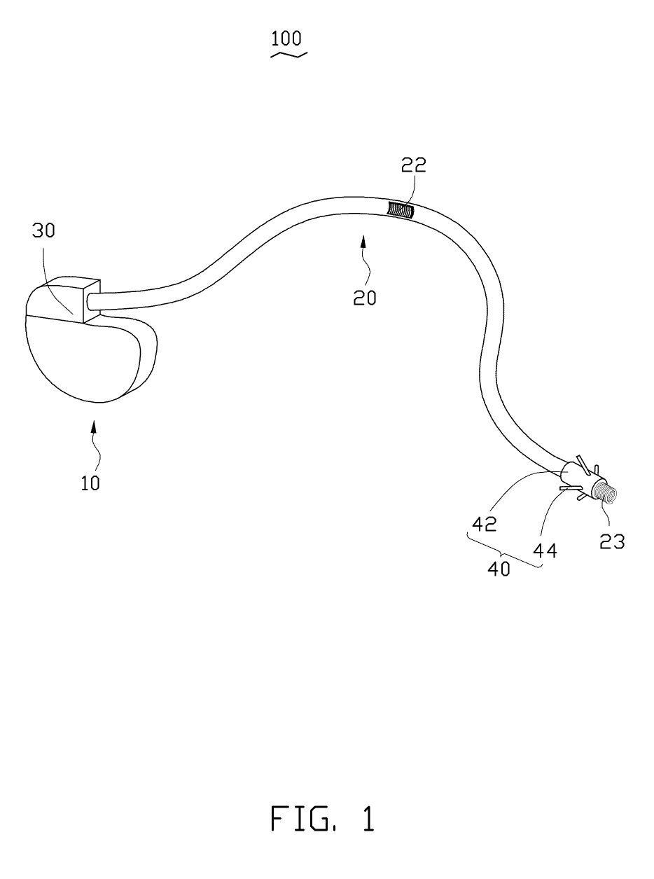

FIG. 1 shows one embodiment of a pacemaker 100 including a pulse generator 10 and an electrode lead 20 electrically connected with the pulse generator 10. The pulse generator 10 can be used to generate pulse signals to stimulate organs of living beings via the electrode lead 20.

The pulse generator 10 can include a shell (not labeled), a power source (not shown), an output circuit (not shown), a sensing circuit (not shown), a control circuit (not shown) and a connector (not shown). The power source, the output circuit, the sensing circuit, and the control circuit are packaged in the shell. A material of the shell can be a biocompatible, corrosion resistant, and deformation resistant metal or alloy. In one embodiment, the material of the shell is titanium (Ti). The power source can provide power for the output circuit, the sensing circuit, and the control circuit. Chemical batteries such as lithium ion batteries can be used in the power source. In one embodiment, the power source includes a lithium-iodine battery. The control circuit is electrically connected with the output circuit and the sensing circuit. The control circuit can control the output circuit and the sensing circuit. The output circuit can be used to generate the pulse signals. The sensing circuit can be used to receive electrical signals generated by the stimulated organs and feed these electrical signals back to the control circuit. The control circuit can adaptively adjust the output signals of the output circuit according to the feedback of the sensing circuit. The connector can be electrically connected with the electrode lead 20. The pulse signals generated by the pulse generator 10 can be transferred to the organ to stimulate the cells. The organs can be a heart, brain, or stomach of living beings.

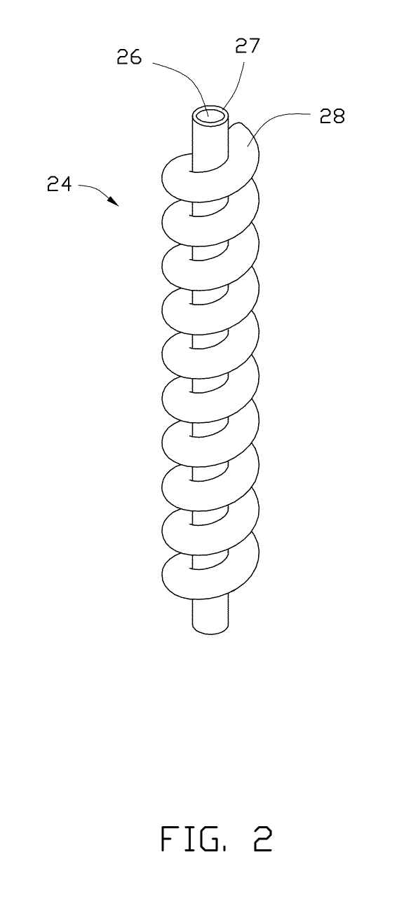

FIG. 1 and FIG. 2 show the electrode lead 20 can include a lead wire 22 and an electrode head 23. The lead wire 22 includes at least one sub-lead wire 24. The sub-lead wire 24 includes a core wire structure 26, a first insulating layer 27 wrapping on an outer surface of the core wire structure 26, and a carbon nanotube composite structure 28 wound around an outer surface of the first insulating layer 27. The electrode head 23 is disposed on one end of the lead wire 22 and electrically connected with the core wire structure 26 of the sub-lead wire 24.

The electrode lead 20 can further include a connecting element 30 and a fastening element 40. The connecting element 30 and the fastening element 40 are respectively disposed on two opposite ends of the lead wire 22 of the electrode lead 20.

The lead wire 22 can be electrically connected with the connector of the pulse generator 10 with the connecting element 30, thereby the core wire structure 26 is electrically connected with the output circuit, and the carbon nanotube composite structure 28 is electrically connected with the sensing circuit. The connecting element 30 can be a hollow cylindrical structure having an external thread. The connecting element 30 is electrically connected with the connector of the pulse generator 10 by the external thread. A material of the connecting element 30 can be a biocompatible, corrosion resistant, and conductive material, such as platinum or platinum-iridium alloy.

The fastening element 40 is fastened on an end of the lead wire 22 having an electrode head 23. The fastening element 40 can be inserted into the human body. The electrode lead 20 is fastened at a predetermined position of the organ, to prevent the electrode lead 20 from slipping. The electrode head 23 is spaced from the fastening element 40. In one embodiment, the fastening element 40 includes a fastening ring 42 and a plurality of fastening wings 44. The fastening ring 42 can be a cylindrical structure. The fastening wing 44 can be a claviform structure extending along a direction away from the central axis of the fastening ring 42. An angle between an axis of the claviform structure and the central axis of the fastening ring 42 can be in a range from about 30 degrees to about 60 degrees. The fastening wings 44 extend away from the lead wire 22, thereby forming a barb structure. The fastening wings 44 can be wrapped with human tissue after being implanted into the human body to fix the electrode lead 20. The fastening element 40 can be made of a biocompatible macromolecule material, such as polyurethane or silicon rubber.

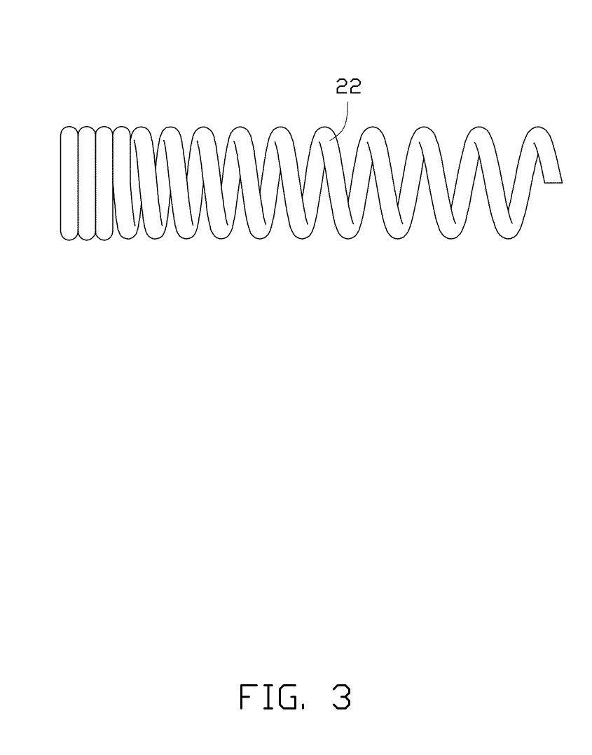

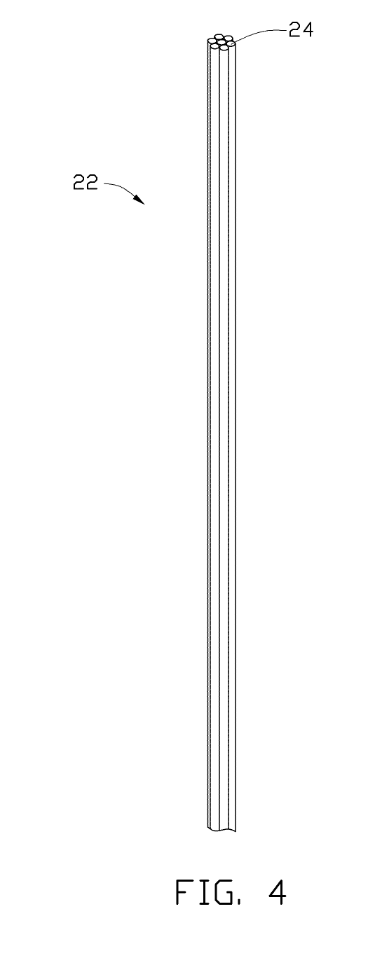

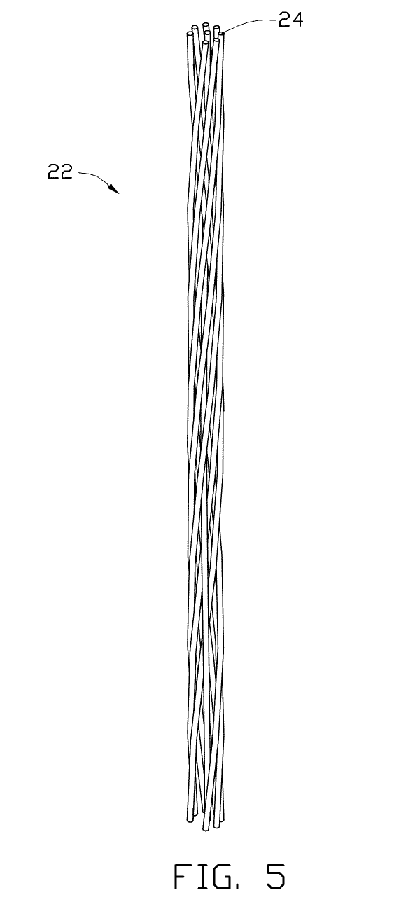

A diameter of the lead wire 22 can be in a range from about 4 millimeters (mm) to about 6 mm. The lead wire 22 can be a linear structure or a helical tubular structure formed by bending the lead wire 22 into a helical shape. FIG. 3 shows the lead wire 22 in a helical tubular structure, which has an excellent elasticity, thereby improving a useful life of the electrode lead 20. FIG. 4 shows the lead wire 22 can be a bundle structure composed of a plurality of sub-lead wires 24 substantially parallel to each other. In another embodiment, the lead wire 22 as shown in FIG. 5 can be a twisted wire structure formed by twisting the plurality of sub-lead wires 24 with each other.

The core wire structure 26 of the sub-lead wire 24 can be made of a material having excellent conductivity, high strength, and toughness, such as stainless steel, carbon fiber, tantalum, Ti, zirconium (Zr), niobium (Nb), titanium alloy, copper (Cu), silver (Ag), platinum (Pt), platinum-yttrium alloy, or platinum-palladium alloy. The core wire structure 26 can have a linear structure or a hollow cylindrical structure formed by spirally bending the linear structure. If the core wire structure 26 is the hollow cylindrical structure, the first insulating layer 27 can wrap on the outer surface of the hollow cylindrical structure. A screw pitch of the spirally bent core wire structure 26 can be in a range from about 0 mm to about 5 mm. In one embodiment, the screw pitch of the spirally bent core wire structure 26 is 0 mm, the material of the core wire structure 26 is Pt, and the first insulating layer 27 wraps on the outer surface of the hollow cylindrical structure.

The carbon nanotube composite structure 28 is wound on an outer surface of the first insulating layer 27. In one embodiment, the carbon nanotube composite structure 28 is spirally wound on the outer surface of the first insulating layer 27. A screw pitch of the spirally wound carbon nanotube composite structure 28 can be in a range from about 0 mm to about 5 mm. In one embodiment, the screw pitch of the spirally wound carbon nanotube composite structure 28 is about 3 mm. The carbon nanotube composite structure 28 includes a carbon nanotube structure composed of a plurality of carbon nanotubes and a metal material layer combined with the carbon nanotube structure. The metal material layer can be coated on a surface of the carbon nanotube structure or a surface of each of the plurality of carbon nanotubes in the carbon nanotube structure. The carbon nanotube structure can include at least one carbon nanotube wire, at least one carbon nanotube film, or any combination thereof. The carbon nanotube wire can be spirally wound on the outer surface of the first insulating layer 27. The carbon nanotube film can be spirally wound on the outer surface of the first insulating layer 27. The carbon nanotube film can also be wrapped on the outer surface of the first insulating layer 27, and an extending direction of the carbon nanotubes in the carbon nanotube film can be substantially parallel to an axis direction of the lead wire 22.

The carbon nanotube film can be formed by drawing a carbon nanotube segment from a carbon nanotube array. The carbon nanotube segment includes a plurality of carbon nanotubes substantially parallel to each other. The carbon nanotube film includes a plurality of carbon nanotubes that can be arranged substantially parallel to a surface of the carbon nanotube film. A large number of the carbon nanotubes in the carbon nanotube film can be oriented along a preferred orientation, meaning that a large number of the carbon nanotubes in the carbon nanotube film are arranged substantially along a same direction. In the carbon nanotube film, an end of one carbon nanotube is joined to another end of an adjacent carbon nanotube arranged substantially along the same direction by van der Waals attractive force. A small number of the carbon nanotubes are randomly arranged in the carbon nanotube film, and has a small if not negligible effect on the larger number of the carbon nanotubes in the carbon nanotube film arranged substantially along the same direction.

In the carbon nanotube composite structure 28, the plurality of carbon nanotube wires can be arranged in parallel to form a bundle structure, or twisted with each other to form a stranded wire structure.

The carbon nanotube wire can be a non-twisted carbon nanotube wire, a twisted carbon nanotube wire, or any combination thereof.

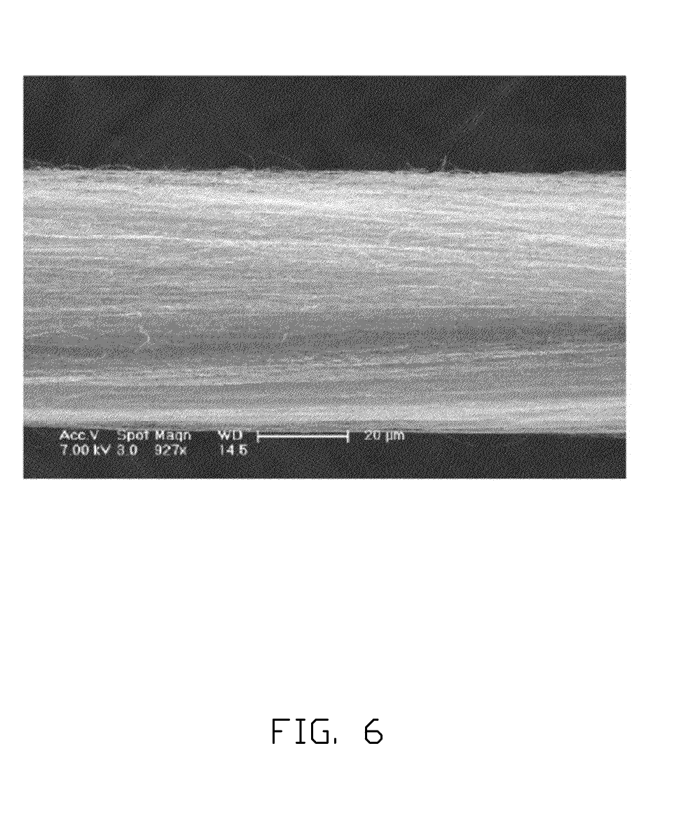

A structure of the non-twisted carbon nanotube wire can be the same as the structure of the above carbon nanotube film having a small width. FIG. 6 shows the non-twisted carbon nanotube wire is a free-standing or stiff structure consisting of a plurality of carbon nanotubes. A majority of the plurality of carbon nanotubes extend in substantially the same direction and parallel to each other. In addition, the majority of the carbon nanotubes are joined end to end by van der Waals attractive forces. Each carbon nanotube in the majority of the carbon nanotubes is joined with the adjacent carbon nanotube lengthwise by van der Waals attractive forces. A minority of the plurality of carbon nanotubes has a random orientation in the non-twisted carbon nanotube wire, and has a very small or negligible effect on the majority of the plurality of carbon nanotubes in view of the arrangement. The non-twisted carbon nanotube wire includes a plurality of successive and preferred-orientation carbon nanotube segments. The plurality of carbon nanotube segments are joined end to end by van der Waals attractive forces. Each of the carbon nanotube segments includes a plurality of carbon nanotubes parallel with each other. The carbon nanotubes in parallel are joined side by side by van der Waals attractive forces. The free-standing or stiff structure of the non-twisted carbon nanotube wire is a result of van der Waals attractive forces acting on the carbon nanotubes joined end to end and side by side. A diameter of the non-twisted carbon nanotube wire can be in a range from about 0.5 nanometers to about 100 microns.

The non-twisted carbon nanotube wire can be formed by the steps of: S(a), choosing a carbon nanotube segment having a predetermined width from a carbon nanotube array by a drawing tool; S(b), moving the drawing tool to pull the chosen carbon nanotube segment at a predetermined speed, thereby pulling out a continuous carbon nanotube wire including the plurality of carbon nanotube segments joined end-to-end by van der Waals attractive forces. Examples of the non-twisted carbon nanotube wire are taught by US Patent Application Publication US 2007/0166223 to Jiang et al.

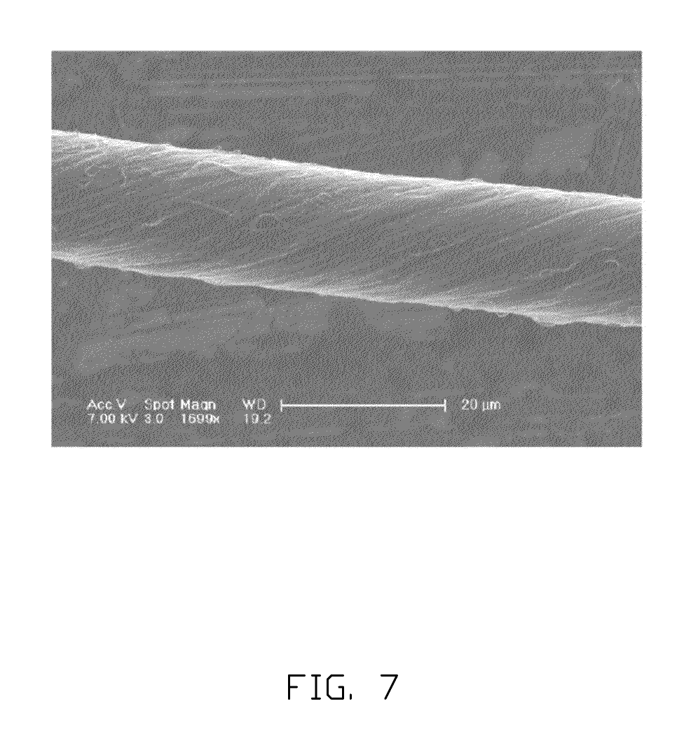

FIG. 7 shows a twisted carbon nanotube wire formed by twisting the non-twisted carbon nanotube wire using a mechanical force to turn the two ends of the non-twisted carbon nanotube wire in opposite directions. The twisted carbon nanotube wire includes a plurality of carbon nanotubes oriented around the central axis of the twisted carbon nanotube wire. The carbon nanotubes are aligned helically around the central axis of the twisted carbon nanotube yarn.

The non-twisted carbon nanotube wire and the twisted carbon nanotube wire can be treated with a volatile organic solvent. After being soaked with the organic solvent, the carbon nanotubes adjacent and substantially parallel to each other in the non-twisted or twisted carbon nanotube wire will bundle together due to the surface tension of the organic solvent when the organic solvent volatilizes. A specific surface area and a viscosity of the carbon nanotube wire will decrease, and a density and strength of the carbon nanotube wire will be increased. A tensile strength of the twisted carbon nanotube wire can be greater than 1200 Mega Pascals (MPa). The tensile strength can reach 1.5 Giga Pascals (GPa), if the diameter of the twisted carbon nanotube wire decreases to 10 microns. The volatile organic solvent can be ethanol, methanol, acetone, dichloroethane, chloroform, and any combination thereof. In one embodiment, the volatile organic solvent is ethanol.

The carbon nanotube film and the carbon nanotube wire can be a pure carbon nanotube structure. The pure carbon nanotube structure consists of pristine carbon nanotubes. The characterization “pristine” signifies that the carbon nanotubes are unfunctionalized or not chemically modified.

The carbon nanotube film, the non-twisted carbon nanotube wire and the twisted carbon nanotube wire have a free-standing structure. The term “free-standing structure” can be defined as a structure that does not have to be supported by a substrate. For example, a free-standing structure can sustain the weight of itself when it is hoisted by a portion thereof without any significant damage to its structural integrity. So, if the carbon nanotube film, the non-twisted carbon nanotube wire or the twisted carbon nanotube wire is placed between two separate supporters, a portion of the carbon nanotube film, the non-twisted carbon nanotube wire or the twisted carbon nanotube wire not in contact with the two supporters, would be suspended between the two supporters and yet maintain film structural integrity. The free-standing structure of the carbon nanotube film, the non-twisted carbon nanotube wire or the twisted carbon nanotube wire is realized by the successive carbon nanotubes joined end to end by Van der Waals attractive force.

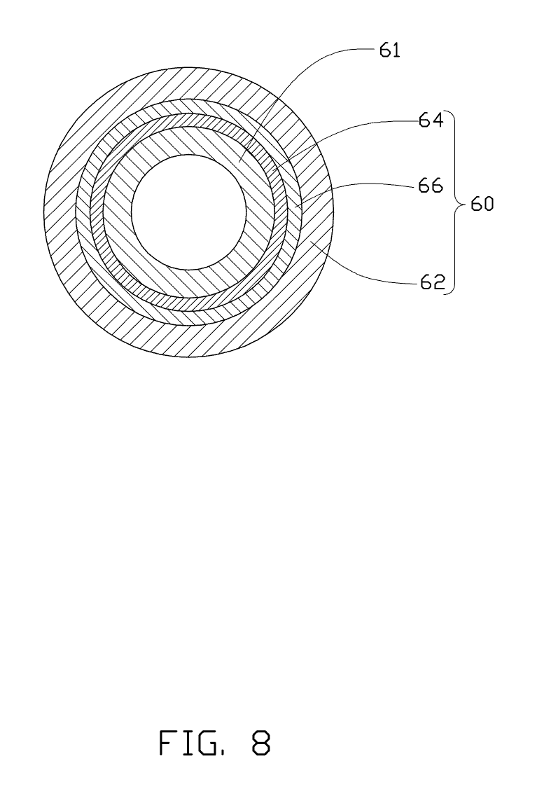

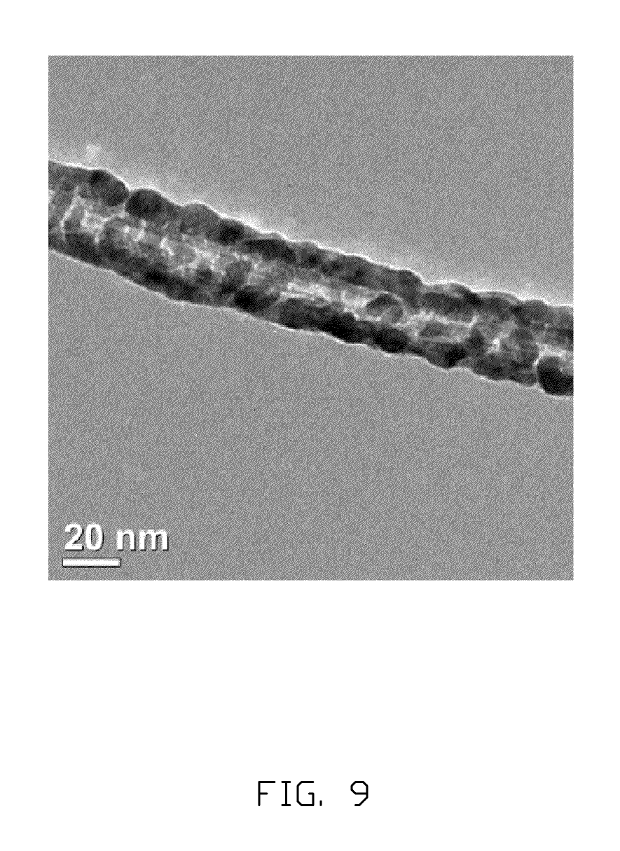

The metal material layer 60 can be coated on the outer surface of the carbon nanotube structure, or the surface of each of the carbon nanotubes of the carbon nanotube structure. In one embodiment, the metal material layer 60 is coated on the surface of each of the carbon nanotubes of the carbon nanotube wire. FIGS. 8 and 9, show the metal material layer 60 can include a wetting layer 64, a transition layer 66 and a conductive layer 62 successively covering on the surface of the carbon nanotube 61. Specifically, the wetting layer 64 covers the circumferential surface of the carbon nanotube 61, the transition layer 66 covers the outer surface of the wetting layer 64, and the conductive layer 62 covers the outer surface of the transition layer 66.

The conductive layer 62 is arranged for enhancing the conductivity of the carbon nanotube composite structure 28. A material of the conductive layer 62 can be at least one of Cu, Ag, gold (Au), or alloys thereof. A thickness of the conductive layer 62 can be in a range from about 1 nm to about 20 nm. In one embodiment, the material of the conductive layer 62 is Au and the thickness is about 2 nm. The carbon nanotube cannot be adequately wetted with most metallic materials, thus, the wetting layer 64 is arranged for wetting the carbon nanotube 61, as well as combining the carbon nanotube 61 with the conductive layer 62. A material of the wetting layer 64 can be at least one of iron (Fe), cobalt (Co), nickel (Ni), palladium (Pd), Ti, and alloys thereof. A thickness of the wetting layer 64 can be in a range from about 1 nm to about 10 nm. In one embodiment, the material of the wetting layer 64 is Ni and the thickness is about 2 nanometers. The transition layer 66 is arranged for combining the wetting layer 64 with the conductive layer 62. A material of the transition layer 66 can be combined with both the material of the wetting layer 64 and the material of the conductive layer 62, such as Cu, Ag, or alloys thereof. A thickness of the transition layer 66 can be in a range from about 1 nm to about 10 nm. In one embodiment, the material of the transition layer 66 is Cu and the thickness is about 2 nm. The transition layer 66 and the wetting layer 64 are optional. If the metal material layer 60 only includes the conductive layer 62, the conductive layer 62 can directly cover the surface of the carbon nanotube 61.

The conductive layer 62, the wetting layer 64 and the transition layer 66 can be formed by a physical method or a chemical method. The physical method can be a physical vapor deposition method such as a vacuum evaporation method or an ion sputtering method. The chemical method can be an electroplating method or a chemical plating method. In one embodiment, the metal material layer 60 is combined with the non-twisted carbon nanotube wire by vacuum evaporation method. In the method, a metal material is vaporized or sublimed to form a metal gas. The metal gas meets the cold carbon nanotube wire and coagulates on the whole circumferential surface of the carbon nanotube wire. The carbon nanotube wire has gaps defined between the carbon nanotubes, and the carbon nanotube wire can be suspended due to the free-standing structure. Thus, the metal material can permeate into the gaps between the carbon nanotubes, thereby depositing on the circumferential surface of each of the carbon nanotubes of the carbon nanotube wire. In addition, the metal material layer 60 is thin, and the gaps remain between the metal material layers 60 on the carbon nanotubes.

The first insulating layer 27 can be made of a flexible biocompatible material, such as silicon, polyurethane, polytetrafluoroethylene, or a copolymer of the silicon and polyurethane. A thickness of the first insulating layer 27 can be in a range from about 1 micron to about 50 microns.

Furthermore, an adhesive layer can be disposed between the first insulating layer 27 and the carbon nanotube composite structure 28. The first insulating layer 27 and the carbon nanotube composite structure 28 can be stably combined together by the adhesive layer. A material of the adhesive layer is not limited. In one embodiment, the material of the adhesive layer is medical adhesive.

In the sub-lead wire 24, the core wire structure 26 can support the carbon nanotube composite structure 28. The carbon nanotubes in the carbon nanotube composite structure 28 has excellent mechanical strength, toughness, and conductivity, and the metal material layer 60 can further improve the conductivity of the carbon nanotube composite structure 28. Therefore, the mechanical strength, the toughness, and the conductivity of the lead wire 22 can be increased by the wound carbon nanotube composite structure 28 on the outer surface of the first insulating layer 27. A working life and sensitivity of the pacemaker can be improved.

The electrode head 23 is electrically connected with the core wire structure 26 of the sub-lead wire 24 and can act as a contact end to directly contact and stimulate the organ of the human body. Specifically, the lead wire 22 can transfer the pulse signals to the organ of the human body by the electrode head 23. The electrode head 23 can be used as a stimulating electrode.

The electrode head 23 can be a common electrode head used in the pacemaker. The electrode head 23 can be fixed to the lead wire 22 by conductive adhesive or welding. A material of the electrode head 23 can be metal or alloy having an excellent conductivity, such as platinum-iridium alloy. A porous material to ensure biocompatibility can be coated on an outer surface of the electrode head 23. In addition, the porous material can increase the contact area between the electrode head 23 and the human body, thereby increasing the sensitivity and sensing efficiency of the pacemaker. The porous material can be activated carbon, carbon fiber, carbon nanotubes, or titanium-nitrogen alloy.

In one embodiment, the electrode head 23 is integrated with the core wire structure 26. A naked end of the core wire structure 26 exposed out from the first insulating layer 27 and the carbon nanotube composite structure 28 and away from the pulse generator 10 can be used as the electrode head 23. In this situation, an additional electrode head 23 disposed on one end of the lead wire 22 is not needed. A length of the electrode head 23 can be in a range from about 0.5 mm to about 2 mm.

Specifically, if the lead wire 22 includes one sub-lead wire 24, the distal end having a linear shape or spiral shape of the core wire structure 26 of the sub-lead wire 24 can be used as the electrode head 23. If the lead wire 22 includes a plurality of sub-lead wires 24, the distal ends of the core wire structures 26 of the plurality of sub-lead wires 24 can extend along different directions to form the electrode head 23 having a radial shape. The distal ends of the core wire structures 26 of the plurality of sub-lead wires 24 can be twisted with each other or substantially parallel to each other to form the electrode head 23 having a linear shape. Furthermore, the electrode head 23 having a linear shape composed of the distal ends of the core wire structure 26 of the plurality of sub-lead wires 24 can be bent to form a spiral shape.

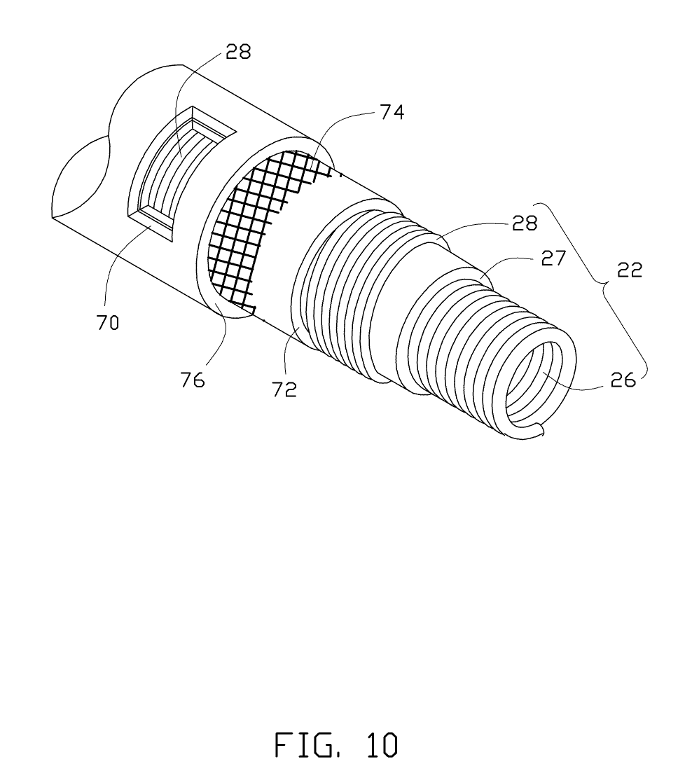

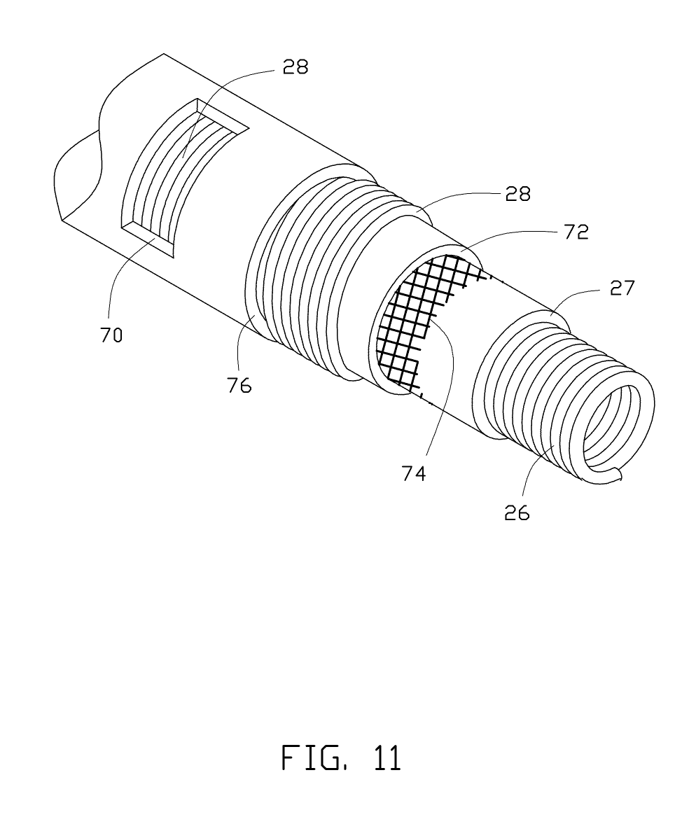

Referring to FIGS. 10 and 11, the electrode lead 20 can further include a second insulating layer 72, a shielding layer 74, and a third insulating layer 76.

The first insulating layer 27 can be coated on the outer surface of the hollow cylindrical structure composed of the spirally bent core wire structure 26. The carbon nanotube composite structure 28 can be spirally wound on the outer surface of the first insulating layer 27. The second insulating layer 72 can be coated on the outer surface of carbon nanotube composite structure 28. The shielding layer 74 can be coated on an outer surface of the second insulating layer 72. The third insulating layer 76 can be coated on an outer surface of the shielding layer 74.

A material of the second insulating layer 72 and the third insulating layer 76 can be the same or different. Specifically, the second insulating layer 72 and the third insulating layer 76 can be made of a flexible biocompatible material, such as silicon, polyurethane, polytetrafluoroethylene, or a copolymer of the silicon and polyurethane. A thickness of the second insulating layer 72 and the third insulating layer 76 can be in a range from about 1 micron to about 50 microns.

A material of the shielding layer 74 can be a conductive material, such as metal or carbon nanotubes. The shielding layer 74 can be a continuous film structure or a network structure. For example, the shielding layer 74 can be a metal film or a carbon nanotube film. In one embodiment, a metal wire spirally wound on the outer surface of the second insulating layer 72 can be used as the shielding layer 74. The shielding layer 74 can prevent the pulse signal transferred by the lead wire 22 from electromagnetic interference.

The third insulating layer 76 can be coated on an outer surface of the shielding layer 74. A biological coating layer (not shown) can be coated on the outer surface of the third insulating layer 76. The biological coating layer can improve the biocompatibility of the lead wire 22. A material of the biological coating layer can be zirconia or zirconium nitride.

In one embodiment, an end of the core wire structure 26 of the lead wire 22 is used as the electrode head 23, and exposed out from the first insulating layer 27, the carbon nanotube composite structure 28, the second insulating layer 72, the shielding layer 74, and the third insulating layer 76.

The second insulating layer 72, the shielding layer 74, and the third insulating layer 76 jointly define an opening 70 to expose the carbon nanotube composite structure 28. The exposed carbon nanotube composite structure 28 can directly contact the diseased organ and sense the current signals of the diseased organ. The sensed current signals can be then transferred to the sensing circuit by the carbon nanotube composite structure 28.

Referring to FIG. 11, in another embodiment, the shielding layer 74 and the second insulating layer 72 are disposed between the carbon nanotube composite structure 28 and the first insulating layer 27. Specifically, the shielding layer 74 is directly coated on the outer surface of the first insulating layer 27. The second insulating layer 72 is coated on the outer surface of the shielding layer 74. The carbon nanotube composite structure 28 is spirally wound on the outer surface of the second insulating layer 72. The third insulating layer 76 is coated on the outer surface of the carbon nanotube composite structure 28. The opening 70 is defined by the third insulating layer 72. The carbon nanotube composite structure 28 is exposed out from the opening 70.

A working process of the pacemaker 100 acting on the heart of a human being is described below. The electrode lead 20 is implanted into the heart, with the electrode head 23 and the exposed carbon nanotube composite structure 28 of the electrode lead 20 contacting the heart. The pulse signals are generated by the pulse generator 12 and transmitted to the electrode head 23 and to stimulate the heart. The exposed carbon nanotube composite structure 28 can sense the current signals of the heart and is electrically connected with the sensing circuit, as which the sensed current signals of the heart can be transferred to the sensing circuit. The control circuit can adjust the pulse signals outputted by the output circuit according to the sensed current signals detected by the sensing circuit.

Finally, it is to be understood that the above-described embodiments are intended to illustrate rather than limit the present disclosure. Variations may be made to the embodiments without departing from the spirit of the present disclosure as claimed. Elements associated with any of the above embodiments are envisioned to be associated with any other embodiments. The above-described embodiments illustrate the scope of the present disclosure but do not restrict the scope of the present disclosure.

What is claimed is:

1. An electrode lead of a pacemaker, the electrode lead comprising:
a lead wire comprising at least one sub-lead wire; and an electrode head disposed on an end of the lead wire;
wherein the lead wire comprises at least one sub-lead wire, the at least one sub-lead wire comprises a core wire structure, a first insulating layer wrapping on an outer surface of the core wire structure, and a carbon nanotube composite structure wound around an outer surface of the first insulating layer, the electrode head is electrically connected with the core wire structure.

2. The electrode lead of claim 1, wherein the carbon nanotube composite structure comprises a carbon nanotube structure and a metal material layer combined with the carbon nanotube structure, the carbon nanotube structure comprising a plurality of carbon nanotubes.

3. The electrode lead of claim 2, wherein the carbon nanotube structure comprises a plurality of carbon nanotube segments joined end to end by van der Waals attractive force, each of the carbon nanotube segments comprises a plurality of carbon nanotubes substantially parallel to each other and joined side by side by van der Waals attractive forces.

4. The electrode lead of claim 2, wherein the carbon nanotube structure is a non-twisted carbon nanotube wire, the plurality of carbon nanotubes of the non-twisted carbon nanotube wire extend substantially along one direction and are joined end to end by van der Waals attractive forces.

5. The electrode lead of claim 2, wherein the carbon nanotube structure is a twisted carbon nanotube wire, the plurality of carbon nanotubes of the twisted carbon nanotube wire are aligned helically around a central axis of the twisted carbon nanotube wire and joined end to end by van der Waals attractive forces.

6. The electrode lead of claim 2, wherein the carbon nanotube structure is at least one carbon nanotube film, the plurality of carbon nanotubes of the at least one carbon nanotube film extend substantially along one direction and are joined end to end by van der Waals attractive forces.

7. The electrode lead of claim 6, wherein an extending direction of the plurality of carbon nanotubes in the carbon nanotube composite structure is substantially parallel to an axis direction of the lead wire.

8. The electrode lead of claim 2, wherein the metal material layer coats on a surface of the carbon nanotube structure or a surface of each of the plurality of carbon nanotubes.

9. The electrode lead of claim 8, wherein the metal material layer comprises a conductive layer, and a material of the conductive layer is selected from the group consisting of Cu, Ag, Au, and alloys thereof.

10. The electrode lead of claim 9, wherein the metal material layer further comprises a wetting layer and a transition layer successively disposed between the carbon nanotube wire and the conductive layer, and the wetting layer is disposed between the carbon nanotube and the transition layer.

11. The electrode lead of claim 10, wherein a material of the wetting layer is selected from the group consisting of Fe, Co, Ni, Pd, Ti, and alloys thereof.

12. The electrode lead of claim 10, wherein a material of the transition layer is selected from the group consisting of Cu, Ag and alloys thereof.

13. The electrode lead of claim 1, wherein the electrode head and the core wire structure are integrative, and the electrode head is at a distal end of the core wire structure exposed out from the first insulating layer and the carbon nanotube composite structure.

14. The electrode lead of claim 1, further comprises a second insulating layer, a shielding layer, and a third insulating layer successively coating the outer surface of the lead wire.

15. The electrode lead of claim 11, wherein the second insulating layer, the shielding layer, and the third insulating layer jointly define an opening, and the carbon nanotube composite structure is exposed out from the opening to sense current signals of a disease organ.

16. The electrode lead of claim 1, wherein the lead wire is a linear shaped structure or spirally bent into a hollow cylindrical structure.

17. The electrode lead of claim 1, wherein the at least one sub-lead wire is a plurality of sub-lead wires compactly arranged in parallel to form a bundle structure or twisted with each other to form a twisted wire structure.

18. An electrode lead of a pacemaker, the electrode lead comprising:
a lead wire comprising at least one sub-lead wire; and
an electrode head;
wherein the at least one sub-lead wire comprises a core wire structure, a first insulating layer coating on an outer surface of the core wire structure, a shielding layer coating on an outer surface of the first insulating layer, a second insulating layer coating on an outer surface of the shielding layer, a carbon nanotube composite structure spirally wound on an outer surface of the second insulating layer, and a third insulating layer coating on an outer surface of the carbon nanotube composite structure.

19. The electrode lead of claim 18, wherein the second insulating layer defines an opening, and the carbon nanotube composite structure is exposed out from the opening to sense current signals of a diseased organ.

20. A pacemaker comprising:
a pulse generator, and
an electrode lead electrically connected with the pulse generator, the electrode lead comprising a lead wire comprising at least one sub-lead wire and an electrode head;
wherein the at least one sub-lead wire comprises a core wire structure, a first insulating layer coating on an outer surface of the core wire structure, and a carbon nanotube composite structure wound around the first insulating layer; the electrode head is disposed on an end of the lead wire and electrically connected with the core wire structure.
